(19) United States
(12) Patent Application Publication
(10) Pub. No.: US 2018/0315507 A1
(43) Pub. Date: Nov. 1, 2018

(54) PREDICTION OF ADVERSE EVENTS IN PATIENTS UNDERGOING MAJOR CARDIOVASCULAR PROCEDURES

(71) Applicant: YALE-NEW HAVEN HEALTH SERVICES CORPORATION, New Haven, CT (US)

(72) Inventors: Bobak J. Mortazavi, New Haven, CT (US); Nihar Desai, New Haven, CT (US); Jing Zhang, New Haven, CT (US); Harlan M. Krumholz, New Haven, CT (US)

(21) Appl. No.: 15/894,040

(22) Filed: Feb. 12, 2018

Related U.S. Application Data
(60) Provisional application No. 62/491,109, filed on Apr. 27, 2017.

(51) Int. Cl. G16H 50/30; G16H 10/60

(52) U.S. Cl. CPC G16H 50/30; G16H 10/60

(57) ABSTRACT

Electronic health records (EHR) provide opportunities to leverage vast arrays of data to help prevent adverse events, improve patient outcomes, and reduce hospital costs. A postoperative complications prediction system is provided that extracts data from the EHR and creates features. An analytic engine then provides model accuracy, calibration, feature ranking, and personalized feature responses. The system allows clinicians to interpret the likelihood of an adverse event occurring, general causes for these events, and the contributing factors for each specific patient.

### RELATED APPLICATIONS

This application claims the benefit of and priority to U.S. provisional application Ser. No. 62/491,109, filed Apr. 27, 2017, the content of which is incorporated by reference herein in its entirety.

### BACKGROUND

The early prediction of potential adverse events in patients has been a primary focus of outcomes research and quality improvement efforts in patient care for heart failure [1], readmissions [2], and a variety of other outcomes [3]. These efforts have focused improving patient care in a wide variety of fields, including in early detection of severe events in infants [4], respiratory complications in surgical patients [5], and blood transfusions in cardiac surgery patients [6], by understanding factors leading to conditions like costly readmissions [7], septic shock [8], and unplanned transfers to the intensive care unit [9]. These targeted models for care can help identify patient risk factors and predictors [10][11] as well as potentially address costs of care [12][13].

One major area of research focuses on surgical complications [14][15] and understanding the risk factors involved[16][17] to predict outcomes [18][19]. In particular, under-standing complications such as the risk of infection [8] and respiratory failure [17][20], and other outcomes post-cardiac procedures is a particular area of focus for care [21][22] and cost [13]. Electronic health records (EHR) have been viewed as an increasingly useful source of data for such outcomes re-search across varying patient cohorts and outcomes predictions [23][24][3]. Research on EHR data has ranged from better patient history representation [25][26] to subtyping patient backgrounds [27] for better precision medicine applications and personalized risk predictions [28][7][10]. Recent efforts have aimed at developing patient condition scores to be used for outcomes modeling cases [29][30]. However, with varying EHR systems and a variety of admissions criteria, it is important to understand the data available for outcomes modeling in specific patient populations.

### SUMMARY

The invention includes an approach for finding important clinical data in an electronic health record that can be used to predict a patient's chance of post-operative complications using her/his pre-operative data. Examples of the invention can provide the main reasons (or contributions) for the prediction to help clinicians and patients discuss the risks and potential alternative strategies.

One embodiment of the invention is a method for predicting a patient's risk of a postoperative complication from a procedure. The method includes receiving, by a system comprising a processor, electronic health records stored in memory. The electronic health records include preoperative categorical and continuous data collected from a present patient before undergoing a procedure. The method further includes converting, by the system, the preoperative categorical data into binary variables according to a first rule. The binary variables represent components of a first vector of data having a first vector length. The method further includes receiving, by the system, the preoperative continuous data converted into a time-series according to a second rule different than the first rule. The time-series represent components of a second vector of data having a second vector length. The method further includes merging, by the system, the present patient's first and second vectors of data to form a third vector of data having a third vector length. The method further includes predicting, by the system, the present patient's risk of a postoperative complication from the procedure based on the third vector using a risk prediction model.

In some examples of the invention, the risk prediction model includes a threshold determined from a receiver operating characteristic (ROC) analysis of the risk prediction model. Predicting the present patient's risk of a postoperative complication can include generating a risk prediction by running the risk prediction model on the present patient's third vector of data. The components of the third vector of data represent preoperative categorical and continuous data collected for the patient before undergoing the procedure. The example methods further include comparing the risk prediction to the threshold and determining whether the present patient is at risk of postoperative complications based on the comparison. For example, the present patient is predicted to have a postoperative complication when the risk prediction is greater than the threshold.

In other examples of the invention, a binary variable missing a value can be replaced with a zero or a “no”.

Some examples of the invention further include generating the risk prediction model. The model generation process includes receiving electronic health records including preoperative categorical and continuous data collected from prior patients who underwent the same procedure as the present patient. The process further includes converting each prior patient's continuous data into training binary variables according to the first rule. The training binary variables represent components of a fourth vector of data having a fourth vector length. The fourth vector length associated with each prior patient and the first vector length associated with the present patient are the same.

The model generation process further includes receiving each prior patient's continuous data converted into training time-series according to the second rule. The training time-series represent components of a fifth vector of data having a fifth vector length. The fifth vector length associated with each prior patient and the second vector length associated with the present patient are the same. The process further includes merging each prior patient's fourth vector of data with the fifth vector of data to form a sixth vector of data having a sixth vector length. The sixth vector length associated with each prior patient and the third vector length associated with the present patient are the same.

The model generation process further includes generating a training dataset based on the sixth vector of data of each prior patient and applying a machine learning technique to the training dataset to generate the risk prediction model. The machine learning technique that is applied can be gradient descent boosting.

Another embodiment of the invention is a non-transitory computer readable medium storing instructions which, when executed by a system comprising a processor, cause the processor to perform operations for predicting a patient's risk of a postoperative complication from a procedure. The performed operations include receiving electronic health records stored in memory. The electronic health records include preoperative categorical and continuous data collected from a present patient before undergoing a procedure. The performed operations include further converting the preoperative categorical data into binary variables according to a first rule. The binary variables represent components of a first vector of data having a first vector length. The performed operations include further receiving the preoperative continuous data converted into a time-series according to a second rule different than the first rule. The time-series represent components of a second vector of data have a second vector length. The performed operations include further merging the present patient's first and second vectors of data to form a third vector of data having a third vector length. The performed operations include predicting the present patient's risk of a postoperative complication from the procedure based on the third vector using a risk prediction model.

Yet another embodiment of the invention is a system having a processor and memory storing instructions that, when executed by the processor, cause the processor to perform operations for predicting a patient's risk of a postoperative complication from a procedure. The performed operations include receiving electronic health records stored in memory. The electronic health records include preoperative categorical and continuous data collected from a present patient before undergoing a procedure. The performed operations include further include converting the preoperative categorical data into binary variables according to a first rule. The binary variables represent components of a first vector of data having a first vector length. The performed operations further include receiving the preoperative continuous data converted into a time-series according to a second rule different than the first rule. The time-series represent components of a second vector of data have a second vector length. The performed operations include further merging the present patient's first and second vectors of data to form a third vector of data having a third vector length. The performed operations include predicting the present patient's risk of a postoperative complication from the procedure based on the third vector using a risk prediction model.

The foregoing embodiments and other examples of the invention are described on the context of the research conducted at the Yale-New Haven Hospital (Y-NHH) in Connecticut U.S.A. The cardiovascular procedures considered for this research were coronary artery bypass grafting (CABG), percutaneous coronary intervention (PCI), and implantable cardioverter defibrillators (ICD). The research focused on the extraction of all data from the time of admission to either the start of the procedure or the end of the first twenty-four hours of admission, whichever came first. This time period has been identified by Y-NHH as useful for understanding patient risk factors and determining potential interventions. The data was extracted for use in a machine learning framework to predict patient risk as well as identify the top factors for that risk. Patients and clinicians can use this risk to make better informed decisions on treatment plans with better knowledge about the risk.

The research has led to the development of a system for identifying patients undergoing major cardiovascular procedures at risk for postoperative respiratory failure or infection, two costly outcomes as identified by at Y-NHH. The system tackles the challenges of extracting data from a production-level electronic health record provided by EPIC [33] and the tasks necessary in manipulating data for use in machine learning analytic tools. Further, after developing models to predict postoperative complications using preoperative data, the system can generate interpretable measures of risk to help identify the risk category of the patient, as well as the contributing features to risk in order to better provide clinicians with information that might help prevent such adverse events, providing a framework for more advanced clinical decision support systems in future studies.

Several works have focused on using EHR data to predict outcomes. In [10], authors investigated the use of EHR data to predict readmissions in heart failure patients. Authors extracted patient information (including age, gender, marital status), specific visit information (date, duration, inpatient or outpatient visit, and source of admission), as well as visit information broken up into categories of patient history, labs, medications, and the attending physicians. Using a lasso technique to select the most relevant binary features for the statistical model, authors were able to achieve an area under the Receiver Operating Characteristic (ROC) curve (AUC) of 0.71 and demonstrate potential cost savings. The inventor similarly examines the details of EHR data. The inventor investigated the use of a lasso technique for feature selection in building a logistic regression model. Given the wide array of data types, it will also employ other methods that are better suited for higher dimensional and varied data types.

Work in [8] developed a real-time risk score for septic shock using EHR data. Using the MIMIC dataset available on PhysioNet, authors extracted suspicion of infection via ICD-9 codes, used a multiple imputation approach for missing information or unknown/censored events, and developed an advanced model based upon Cox proportional hazards and lasso regularization for estimating risk. The inventor approached the prediction problems similarly, outlining the data extraction and developing a method to generate predictions; however, because the inventor aims to evaluate predictions at a specific time, the methods used are varied for this purpose, to leverage the cross-sectional data since continuous data as in MIMIC is usually restricted to intensive care units.

The Rothman Index, by PERAHEALTH, is a patient condition score based upon EHR data [29]. This score is built off of 26 variables extracted from medical record data for patients during hospital admissions. In particular, the variables are broken up into vital signs, laboratory tests, cardiac rhythm information, and a variety of nursing assessments that are converted into met/unmet variables [29]. The design of the score was to help quantify patient condition based upon data generated by nurses during admissions.

There are two predictive models developed using the Rothman Index as the primary feature [31][32]. Work in [31] developed a predictive model for unplanned 30-day readmissions using the Rothman Index at discharge, age, gender, insurance type, and service type (medical or surgical). A logistic regression model built from this data had an AUC of 0.73 and the Rothman Index score was shown to be correlated to higher odds of readmission, with an AUC of only 0.68 when the Rothman Index was removed. However, by removing the Rothman Index, the model is left with only the service type for the clinical information. The inventor also considered the effectiveness of the Rothman Index as a way to summarize EHR data in a meaningful manner, but will compare it with use of other clinical data extracted from the medical records.

Work in [32] used the Rothman Index to predict unplanned surgical intensive care unit readmissions, by evaluating the range of Rothman Index scores generated during stays and correlating them to the transfers. However, while evaluating the importance of first and last Rothman Index scores, no predictive models were built to consider the effects of a variety of Rothman Index scores throughout the patient encounter to predict adverse events. The inventor developed predictive models for post-surgical outcomes through a variety of modeling techniques based upon increased Rothman Index data availability and increased EHR data availability.

### BRIEF DESCRIPTION OF THE DRAWINGS

The foregoing and other objects, features and advantages will be apparent from the following more particular description of the embodiments, as illustrated in the accompanying drawings in which like reference characters refer to the same parts throughout the different views. The drawings are not necessarily to scale, emphasis instead being placed upon illustrating the principles of the embodiments.

FIG. 1A continuing on FIG. 1B is a diagram of electronic health records (ERH) data organized into tables.

FIG. 2 is a system diagram of a data analytic engine in accordance in an example embodiment of the invention.

FIG. 3 is a chart showing percutaneous coronary intervention (PCI) patient observed respiratory failure rate per quartile of risk.

FIG. 4 is a flowchart of an example process for identifying patients undergoing cardiovascular procedures at risk for postoperative complications in accordance in an example embodiment of the invention.

FIG. 5 is Table I, showing data organized in tables.

FIG. 6 is Table II, showing the event rates of respiratory failure and infection.

FIG. 7 is Table III, showing the best single model AUC and the model type that generated it for each test type and patient cohort.

FIG. 8 is Table IV, showing the best mean AUC's and corresponding models for predicting respiratory failure in CABG patients.

FIG. 9 is Table V, showing the best mean AUC's and corresponding models for predicting respiratory failure in PCI patients

FIG. 10 is Table VI, showing the best mean AUC's and corresponding models for predicting respiratory failure in ICD patients.

FIG. 11 is Table VII, showing the best mean AUC's and corresponding models for predicting infection in CABG patients.

FIG. 12 is Table VIII, showing the best mean AUC's and corresponding models for predicting infection in PCI patients.

FIG. 13 is Table IX, showing the best mean AUC's and corresponding models for predicting infection in ICD patients.

### DETAILED DESCRIPTION

Section I. Method

The disclosure details the personalized predictions of postoperative complications in cardiovascular procedure patients. It also covers the extraction of data from the EPIC electronic health record system [33] used by Yale-New Haven Hospital (Y-NHH). The cohort consisted of patients admitted to the Heart and Vascular Center (HVC) for cardiac procedures, with a primary principal procedure code for CABG, PCI or ICD. This study used all data available in the EHR from February, 2013 (the go-live date for EPIC at Y-NHH) through September, 2015. As prior data were stored on a different HER system, all visits from this date forward were considered first visits. Methods considered for this work considered data upon patient presentation at admission and collected from then forward. As a result, no outpatient data, including emergency room visit data that led to the admission was included, except for the source of admission, to understand the transfer-in status of the patient. For each patient, if multiple visits occurred, only the first visit was considered, though the lack of prior visit data lends the methods developed to repeated use.

Outcomes of respiratory failure and infection were defined by the QUALITY VARIATION INDICATORS (QVI's) developed by Yale-New Haven Hospital to identify those patients with adverse events developed postoperation, which result in poor patient outcomes and extensive cost to the medical system [13][34]. 111 patients passed away after the procedure, with only 46 being within 48 hours of procedure.

A. Data Source:

Data were extracted for each admission. Each visit's dataset consisted of data from admission time to either 24 hours or the start of patient's first procedure, whichever came first; this period of time was believed to be long enough to gather clinically relevant information on the patients to provide an understanding of patient risk prior to the procedure that resulted in the adverse event. Further, this aligned with clinical rounds typically happening every morning and procedures often happening soon after admission. The desired goal, therefore, was to create a dataset and system that would serve as a balance between early enough for appropriate decision making and late enough for considering a wide array of data. The following categories of information were gathered:
Patient Information: Included features, such as age, gender, insurance, and admission information.
Patient History: Included information, such as the patient problem list and admission diagnosis codes (ICD-9).
Visit Information: Included primary principal procedure information, admission time, and attending staff information.
Medical Information: Included medications prescribed, laboratory results, and patient vitals, including temperature, pulse oxygenation, systolic blood pressure, diastolic blood pressure, respiratory rate, and heart rate.
Rothman Index: Rothman Index scores.

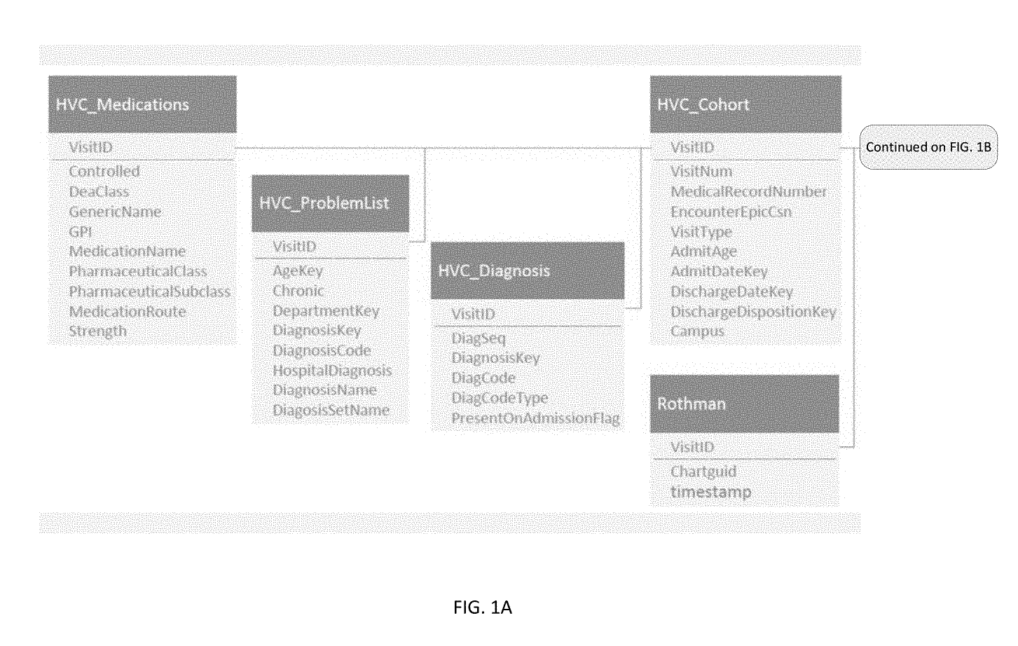

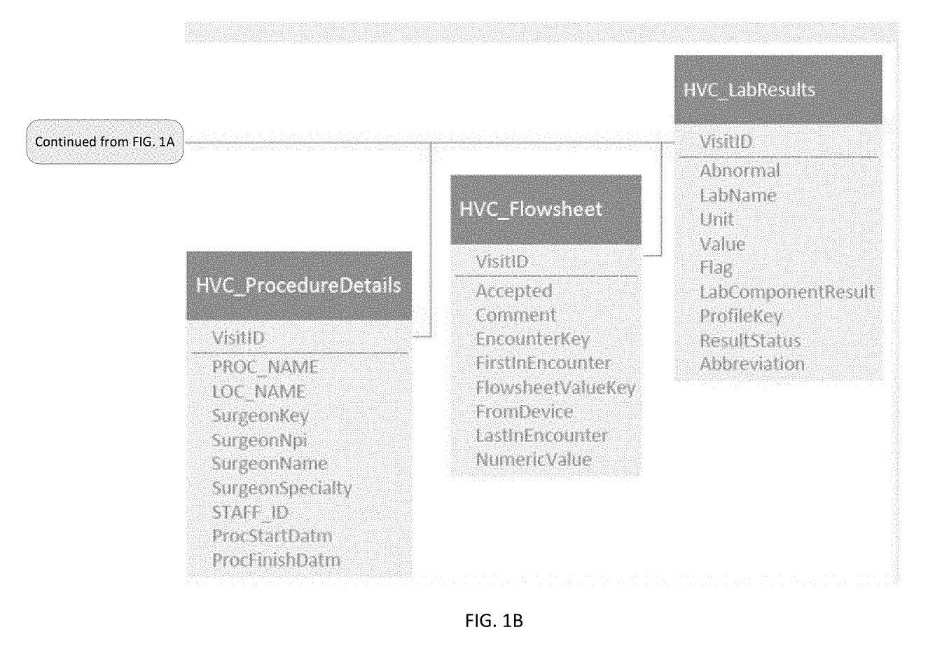

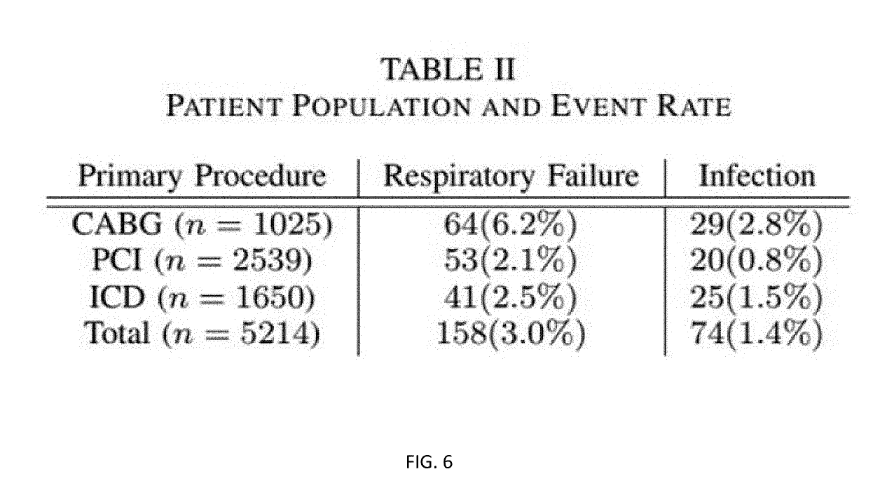

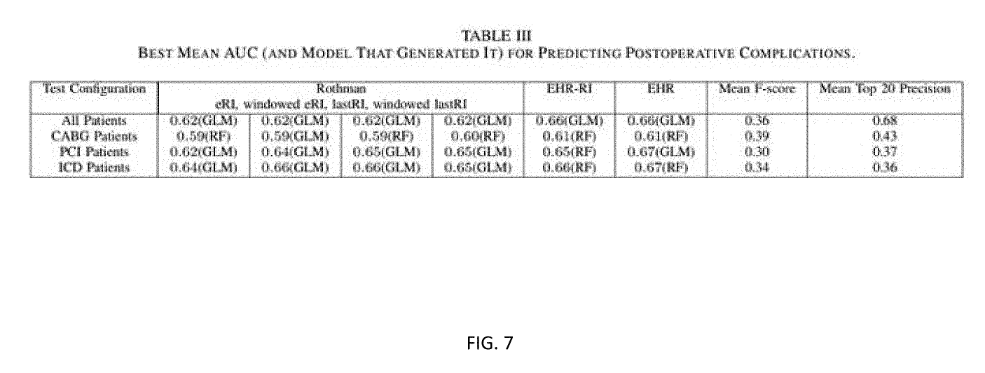

The data were extracted from the EHR data tables shown in FIG. 1, where each VisitID in the patient cohort table had a one-to-many relationship with entries in each of the other tables of the database. The data were organized in seven tables (plus a Rothman Index Scores table), listed in Table I (see FIG. 7). These tables were joined from back-end tables storing data from the front-end of EPIC. The Cohort table contained patient information, including the admission source (e.g. self-referral, transfer from another hospital, transfer from another unit, physician referral), insurance information (e.g. Medicare, private insurance, etc.), and personal information (e.g. age, gender, race if provided). The patient population included 1025 CABG patients, 2539 PCI patients, and 1650 ICD patients. Table II (see FIG. 6) shows the event rates of respiratory failure and infection. Despite the low event rates, these patients were adversely harmed and attributed a significant cost to the hospital [13]. The data extracted were structured data organized in the back-end data warehouse for the EHR system, allowing for quick manipulation of fields for feature extraction.

B. Feature Extraction:

Once the appropriate data were extracted from the EHR, it needed to be converted into a format suitable for use in machine learning analytics. Much of the information was stored in a one-to-many format needing manipulation. For example, in FIG. 1, medication information was stored in a fashion where a single VisitID might consist of multiple rows in the database, where the medication name and pharmaceutical class fields contained each prescribed medication information.

All categorical variables were created into distinct binary yes/no variables for each factor. For example, the problem list and diagnosis information for each visit were converted into a series of binary yes/no variables for each individual ICD-9 code, lab results had a yes/no for lab conducted and results available. The yes/no variable allows the machine learning algorithm to understand if the remaining extracted lab variables, namely, numeric results and alert flags (based upon stored reference values); were missing values or reported results from a conducted lab.

The flowsheet table shown in FIG. 1 contained many of the structured vital sign information for each patient. As vitals may have been taken multiple times between admission and procedure start time, a time-series was generated for each variable, as was for the Rothman Index. Features for the length of the time-series as well as the mean, standard deviation, minimum, and maximum were created as well. Because this created variable-length time-series, each patient's first and last readings were saved, the windowed features calculated, and additional readings were dropped, rather than determine an appropriate imputation. More complex methods might find spurious patterns in the specific readings if improperly imputed. Time-series data were represented by first reading, last reading, number of readings, mean, minimum, maximum, and standard deviation. The foregoing representations of time-series data is a non-limiting example and can include other representations like variance and the number of peaks. For laboratory readings, only the last laboratory reading was considered due to the sparse nature.

1) Grouping of Variables:

The extraction of the dataset resulted originally in 14353 variables per patient. This set of features included 1764 prior history variables and diagnosis codes, 8328 variables for laboratory information, 1942 variables for medication information, and 2319 variables for patient admission information. Thus, some dimension reduction was performed. The machine learning methods used (discussed below in Section I-D) were selected because of their abilities to select a sparse set of features from a high-dimensional set such as this. Preliminary dimension reduction, however, could be done manually by changing the specificity of the features created. Taking guidance from medical expertise as well as national registries such as the National Cardiovascular Data Registry (NCDR) [35], features were merged whenever clinically appropriate. For example, the 1577 binary variables from medication/dosage information were reduced to 295 variables of medication counts via the use of pharmaceutical class. More explicitly, rather than have a variable for each dosage of aspirin given (e.g., 125 mg vs. 165 mg), these were combined into a variable that includes just aspirin, and this was combined further to the pharmaceutical class of all the medications. Similar techniques were applicable to the insurance information, race information, and laboratory information. Prior history variables were grouped together when known chronic condition flags were met. This reduced medication to 295 variables, grouped prior history variables, laboratory, and others as well, by eliminating those with no variance. This reduction of variables resulted in a final set of 9828.

2) Missing Variables:

The potential for missing data after extraction is an important issue in EHR datasets. Data might be missing for a variety of reasons, from the patient choosing not to disclose race information to laboratory results that were normal did not set the flag variables, and are dependent upon the implementation strategy and completeness in filling out the interactive forms and transmitting that data to the backend databases. In many cases, binary indicator variables were imputed with a 0/no if not present for a given visit (i.e., 0 indicates either missing or not prescribed medication, 1 is a definitive prescription of a medication). For any missing variable that could not similarly be coded, such as numeric vital sign information as well as Rothman Index, it was determined that missing data should be imputed with the mean value, because a 0 Rothman Index score, for example, would indicate a severely ill patient. This imputation occurred after the training sets and testing sets were created, using only the training means, so that no knowledge of the testing data was included in this calculation.

3) Normalization:

After the dataset is created, it was z-scored (centered and scaled) by subtracting the feature mean and dividing by the feature standard deviation. If the feature standard deviation was 0 the feature was removed entirely.

C. Validation:

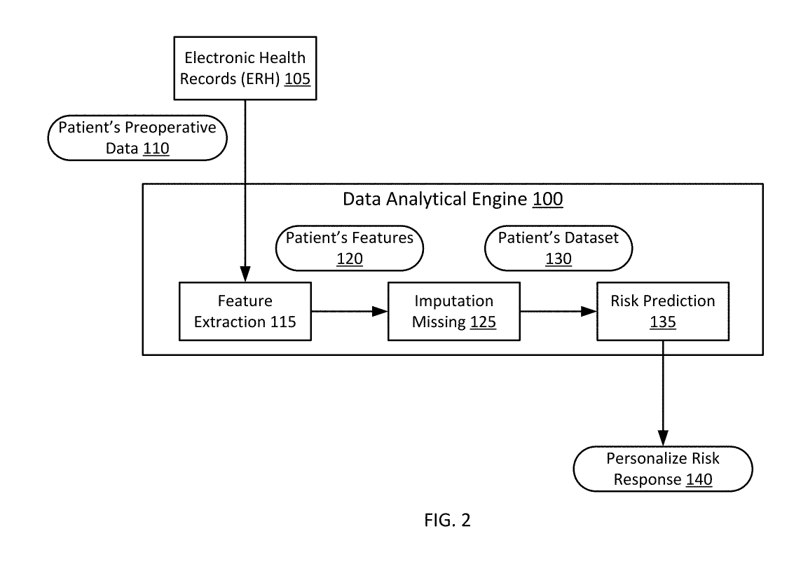

A cross-validation framework was setup to analyze the effectiveness of the proposed methods. Many clinical papers often use a single 80/20 random split to create their training and testing datasets [2][1]. The inventor used a five-fold stratified cross-validation in order to create similar 80/20 splits and maintain the observed event rate in each fold. The imputation steps as well as the normalization, indicated above, were carried out after the folds were created, with the training means being used to impute both the training set and the testing set alike, and the training means and standard deviations being used to normalize the training set and the testing set. The system layout for validation is shown in FIG. 2.

D. Data Analytic Engine:

Once the training set was created, it was passed to three different modeling techniques. Those techniques were logistic regression with lasso regularization (a form of generalized linear model), random forest, and gradient descent boosting. The analysis was carried out in R, with the glmnet package being the chosen implementation for the logistic regression and generalized linear model approach (hereinafter GLM) [36], the randomForest package for the random forest algorithm (hereinafter RF)[37], and xgboost or eXtreme Gradient Boosting package as the implementation of a gradient descent boosting method chosen (hereinafter XGB) [38] respectively. These techniques were selected due to their ability to select a sparse set of features while training, to avoid overfitting, and further reduce the dimensionality of the problem, where applicable. Further, GLM is commonly used in clinical practice and outcomes research, linking to similarity in related works, while RF and XGB are particularly good at dealing with data of mixed types such as these by setting differing thresholds in each particular decision tree. Further, as these last two techniques are non-linear methods, they might provide stronger results than linear methods commonly used in clinical outcomes research.

1) Hyperparameter Tuning:

For GLM, an internal cross-validation on the training data was run in order to tune the algorithm hyperparameters, with the area under the receiver operating characteristic curve or “AUC” being the optimized measure. Sample weights were provided, where the weight for each adverse event example was the ratio of dataset size to number of adverse outcomes (the inverse of the event rate). The default parameters were selected for RF; and XGB was tuned using a grid-search for the number of iterations (100 to 1000 in 100 step-size increments) and the maximum depth of each tree (5 to 10) in an internal cross-validation.

E. Prediction:

Models were trained on the entire dataset as well as created by patient cohort and outcomes splits. Once trained, each algorithm generated a response for the test set. This response was a generated probability of a postoperative complication, rather than a strict label output. From this, a receiver operating characteristic curve (ROC) curve plot allowed calculation of an AUC. AUCs are often reported in clinical prediction models [1], due to the measure being unaffected by class imbalance [39]. However, to understand how such models would be used prospectively, more information should be presented regarding the predictive accuracy. After the models and AUCs were generated, an optimal threshold probability was selected to generate the classification labels. The threshold selected was that which maximized the F-score. From this classification, the true positives, true negatives, false positives, and false negatives were calculated and from that an F-score. Finally, a further metric was calculated regarding the precision of the top 20 predictions, to see if all the true positives are captured in the riskiest patients predicted as a numeric measure for how well the algorithm is calibrated. The 20 were selected based upon the total number of adverse events in each sub-group, knowing that a subset of these would exist in each fold, and to evaluate if creating a larger interval would account for all the true positives or not. This value can be altered to highest deciles of risk, quartiles, and the definitions should be created in consultation with the clinical professionals involved to understand their desires of evaluating ‘high-risk’ patients. For all the measures, the mean and 95% confidence intervals were calculated. Calibration plots were also created for the best models generated.

F. Personalized Risk Factors:

The ability to interpret model predictions is highly desirable for clinicians, and to potentially help determine risk factors resulting in the prediction and potentially helping determine interventions or actions that might prevent the postoperative complication. While the models provided the selected global features, feature importance was extended to provide patient-specific results. Namely, GLM provided a vector of \vec{β}=(β₁, β₂, . . . ) coefficients for each parameter, which provide the global feature importance and where the length of the vector is equal to the number of features (and a large number are 0 for non-selected features). For every test patient x̅ [symbol] [symbol] x₁, x₂, . . . [symbol] the component-wise multiplication of the two vectors results in a feature-contribution vector \vec{feat}=(β₁×x₁, β₂×x₂, . . . ) whose components are then summedtogether by GLM for the resulting prediction. Sorting these components then provided the clinicians with the top contributing factors of risk for each individual patient.

Section II. Results

A. Test Framework:

The analysis presented in Sections I-D, I-E, and I-F above was run on the five-fold cross-validation dataset. As a reminder, all data were used from the admission time until either the first procedure start or 24 hours, whichever came first. All time-series based features used considered all available data in this window. In order to evaluate the effectiveness of all the features generated from the EHR, and to compare against methods previously generated using the Rothman Index [31][32], the following four Rothman tests [31][32] as well as two configurations with the data extracted in this disclosure, were created, over the same extraction window as the remaining data:
Rothman Index test using patient demographics, history, insurance, and the earliest Rothman Index—hereinafter ‘eRI’
Rothman Index test using eRI as well as mean, standard deviation, minimum, and maximum—hereinafter windowed ‘ eRI’
Rothman Index test using patient demographics, history, insurance, and the latest Rothman Index—hereinafter ‘lastRI’
Rothman Index test using lastRI as well as mean, standard deviation, minimum, and maximum—hereinafter ‘windowed lastRI’
EHR dataset—all extracted features without the Rothman Index features—hereinafter ‘EHR-RI’
Complete EHR Dataset—all extracted features including the Rothman Index features—hereinafter ‘ERH’

B. Single Model Tests:

The first tests designed were run in order to validate the effectiveness of separating patients by procedures as well as outcome. Table III (see FIG. 7) shows the best single model AUC and the model type that generated it for each test type and patient cohort. Further, the final two columns show the mean F-score and mean precision of the top 20 generated risk scores. While the top 20 precision is likely increased due to the larger number of cases to train and test on, the lower AUC indicates that only the highest risk is well identified. Indeed, the similar F-scores show that, even with high precision, recall is affected, and that only the highest risk patients are well identified. It became clear that some prediction results were strengthened by specifying the patient population, likely due to the different risks associated with each procedure type. The remainder of the tests evaluated the hypothesis that multiple models should be developed for the prediction of postoperative complications for the patient procedures due to the patient heterogeneity in each case.

C. Respiratory Failure:

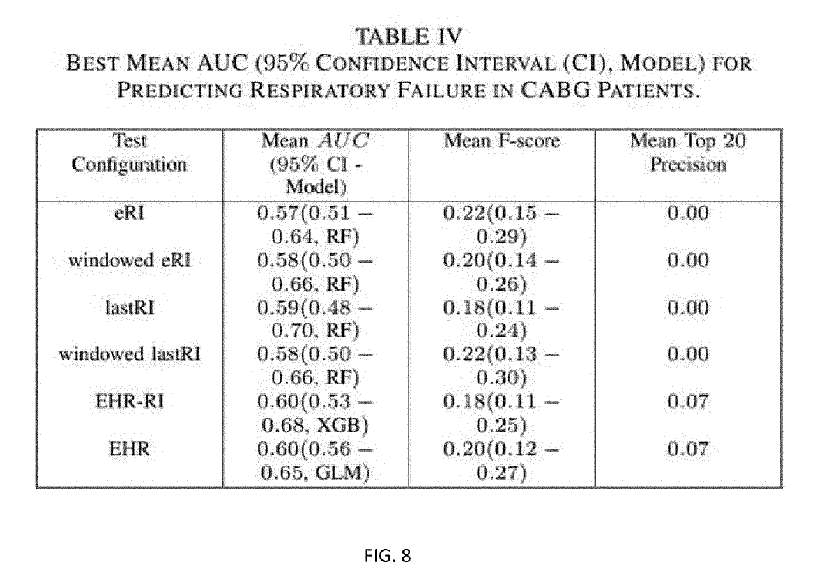

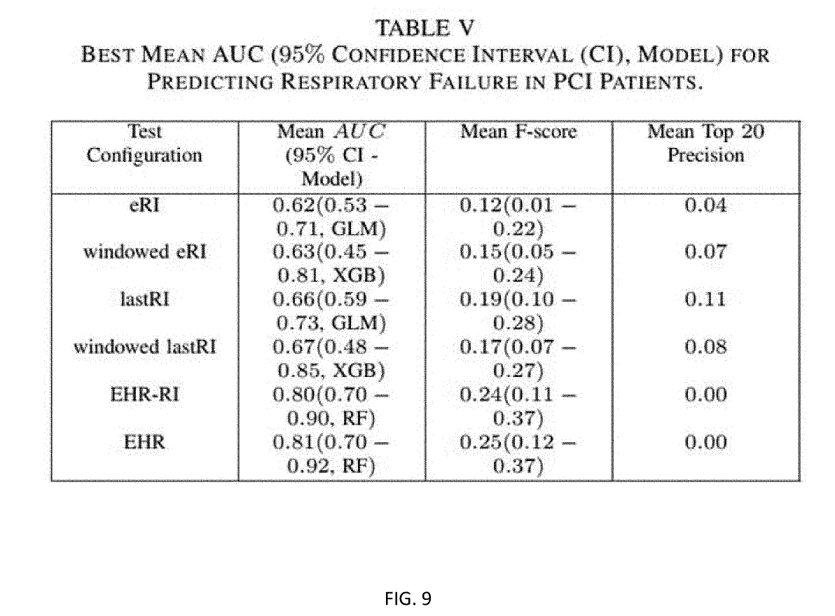

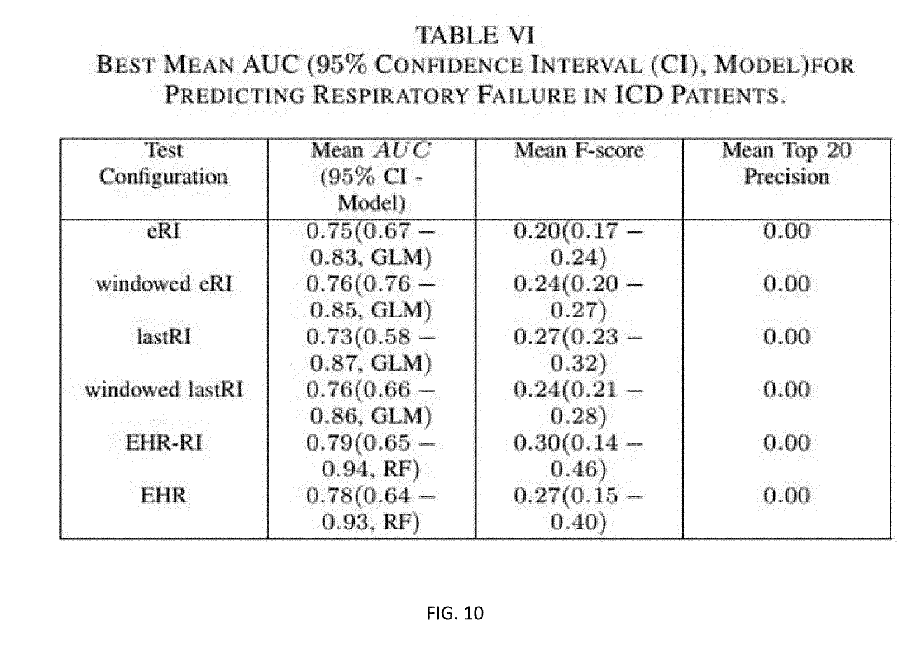

Models were created separately for coronary artery bypass grafting (CABG) patients, percutaneous coronary intervention (PCI) patients, and implantable cardioverter defibrillators (ICD) patients to predict respiratory failure. The results for each can be found in Table IV (see FIG. 8), Table V (see FIG. 9), and Table VI (see FIG. 10), respectively. For each test case, GLM, RF, and XGB models were created, with the mean AUC and mean F-score of the strongest model over cross-validation presented. The mean precision of the top 20 predicted risks are also presented to present an interpretation of model calibration independent of the cutoff threshold selected to generate the F-score. This means that, for the top 20 patients when sorted by outputted risk score, the precision was then calculated on these patients only.

1) CABG Patients:

Note that for CABG patients, in Table IV (see FIG. 8), using the windowed information of the Rothman Index provided a higher AUC (mean AUC's of 0.59 and 0.58 for windowed eRI and windowed lastRI, respectively). Using the last Rothman Index helped provide higher F-score for an F-score of 0.22 for windowed lastRI. In all cases, the use of EHR data provided a higher AUC (0.60 for both cases) but a slightly lower F-score (0.18 and 0.20 for EHR-RI and HER, respectively). The EHR-RI and EHR had a more defined high-risk group with the top 20 measure of 0.07 in both cases. While the best CABG model was GLM, the similar AUC across each data configuration and each method indicates that linear models performed sufficiently well. For the model with the highest F-score, the EHR model, the top features selected in each fold are listed here:
Fold 1: Respiration Rate, Prior History: Hypovolemia, Lab: Blood Urea Nitrogen (BUN) is High, Primary Diagnosis: Coronary Atherosclerosis of Native Coronary Artery
Fold 2: Prior History: Hypovolemia, Lab: Prothrombin Time is Abnormal, Lab: MCH is unspecified
Fold 3: Earliest Respiration Rate, Lab: Albumin, Prior History: Hypovolemia, Lab: Albumin
Fold 4: Earliest Heart Rate, Prior History: Hypovolemia, Lab: PO2 Arterial, Med: Serotonin-2 Antagonist, Patient Demographics: Race—Other, Primary Diagnosis: Coronary Atherosclerosis of Native Coronary Artery
Fold 5: Prior History: Other or Unspecified Hyperlipidemia, Primary Diagnosis: Coronary Atherosclerosis of Native Coronary Artery

As described in Section I-B above, the flags and thresholds are predetermined by the laboratory and defined within the table in EPIC.

2) PCI Patients:

All models for PCI patients, presented in Table V (see FIG. 9), were able to better predict respiratory failure than in CABG patients or in ICD patients. Similar to CABG patients, using the windowed information of the Rothman Index provided a higher AUC than the single measure (mean AUC's of 0.63 and 0.67 for windowed eRI and windowed lastRI, respectively). Using the last Rothman Index helped provide higher F-score for an F-score of 0.19 for lastRI. In all cases, the use of EHR data provided significantly higher AUC measurements from both the single model for PCI patients (0.67) and any of the Rothman Index test cases, with an AUC of 0.80 for EHR-RI and 0.81 for EHR. Similarly, the F-score for these two cases were higher as well, at 0.24 and 0.25, respectively. However, none of the cases performed well in the top 20 precision measure. For the model with the highest F-score, the EHR model, the top features are listed here:
Fold 1: Prior History: Acute Respiratory Failure, Med: Analgesics Narcotic-Anesthetic Adjunct Agents, Lab: ECG—P Axis, Lab: Glucose Meter is Low, Prior History: Acute Myocardial Infarction of Inferolateral Wall Episode of Care Unspecified
Fold 2: Med: Analgesics Narcotic-Anesthetic Adjunct Agents, Med: IV Solutions Dextrose Water, Prior History: Acute Respiratory Failure, Admit Source: Self Referral, Lab: MCHC
Fold 3: Med: Analgesics Narcotic-Anesthetic Adjunct Agents, Prior History: Acute Respiratory Failure, Lab: ECG—P Axis, Lab: CO2, Lab: Glucose Meter is Low
Fold 4: Prior History: Acute Respiratory Failure, Lab: CO2, Prior History: Cardiogenic Shock, Lab: MCHC, LAB: Bun to Creatinine Ratio
Fold 5: Med: Analgesics Narcotic-Anesthetic Adjunct Agents, Med: IV Solutions Dextrose Water, Lab: Glucose Meter is Low, Lab; B-type Natriuretic Peptide ProBNP is Abnormal, Lab: Bands Present is Abnormal

3) ICD Patients:

ICD patient respiratory failure predictions, presented in Table VI (see FIG. 10), were improved over the single model AUC of 0.67 from Table III (see FIG. 7). The Rothman Index models performed better than the single model case, as well, with the windowed eRI and windowed lastRI each achieving the higher AUC of 0.76. Using the last Rothman Index score improved the F-score of the models to 0.27. The EHR-RI and EHR models performed the best, with the RF models achieving AUC's of 0.79 and 0.78, respectively and F-scores of 0.30 and 0.27, respectively. For the model with the highest F-score, the EHR-RI model, the top features are listed here:
Fold 1: Prior History: Acute Respiratory Failure, Primary Diagnosis: Acute on Chronic systolic (Congestive) Heart Failure, Primary Diagnosis: Combined Systolic and Diastolic Heart Failure—Acute on Chronic, Admit Source: Self Referral, Med: Sodium-Saline Preparations
Fold 2: Primary Diagnosis: Systolic Heart Failure-Acute on Chronic, Prior History: Acute Respiratory Failure, Admit Source: Physician or Clinical Referral, Admit Source: Self Referral, Lab: Glucose Meter
Fold 3: Prior History: Acute Respiratory Failure, Primary Diagnosis: Systolic Heart Failure—Acute on Chronic, Admit Source: Self Referral, Primary Diagnosis: Combined Systolic and Diastolic Heart Failure—Acute on Chronic, Lab: Lactate
Fold 4: Admit Source: Self Referral, Admit Source: Emergency, Primary Diagnosis: Systolic Heart Failure—Acute on Chronic, Prior History: Intermediate Coronary Syndrome—Unstable Angina, Lab: ECG T Wave Axis
Fold 5: Prior History: Acute Respiratory Failure, Primary Diagnosis: Systolic Heart Failure-Acute on Chronic, Admit Source: Self Referral, Primary Diagnosis: Combined Systolic and Diastolic Heart Failure—Acute on Chronic, Lab: Potassium is High Panic

D. Infection:

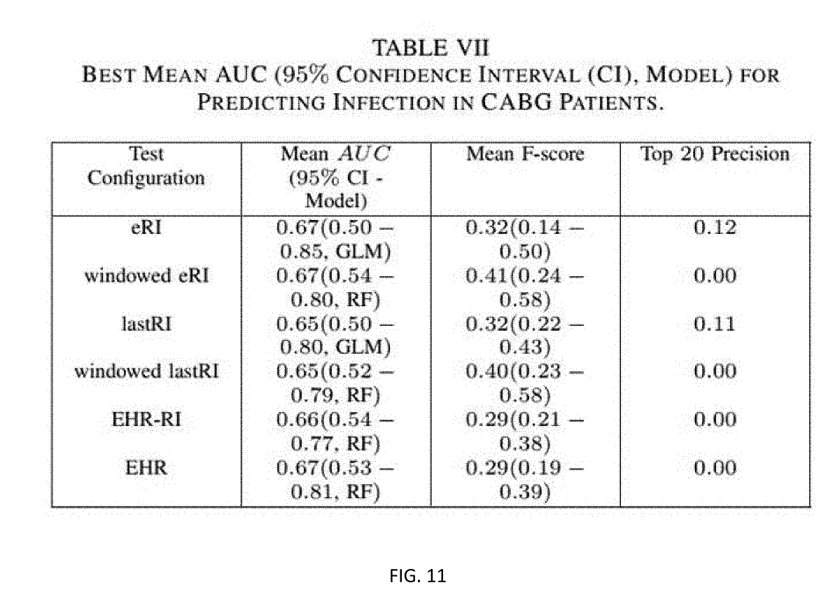

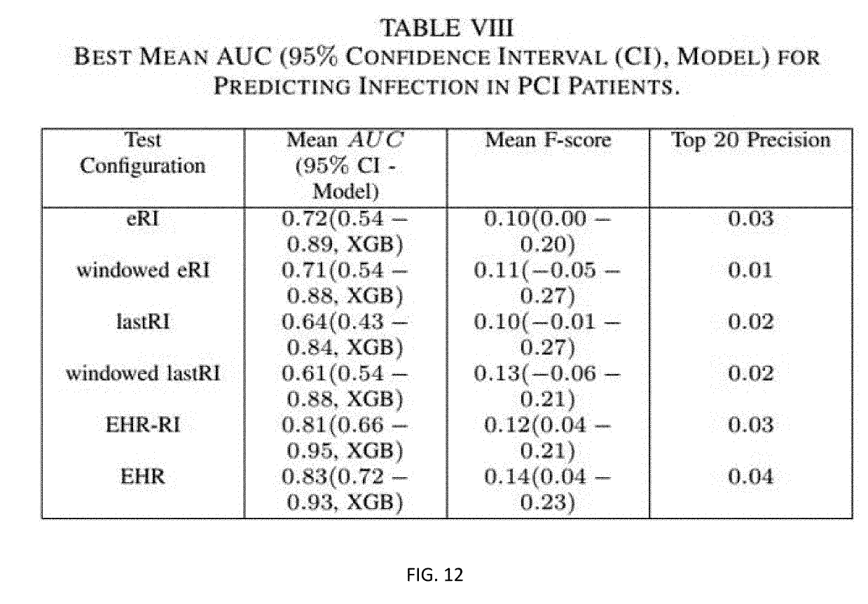

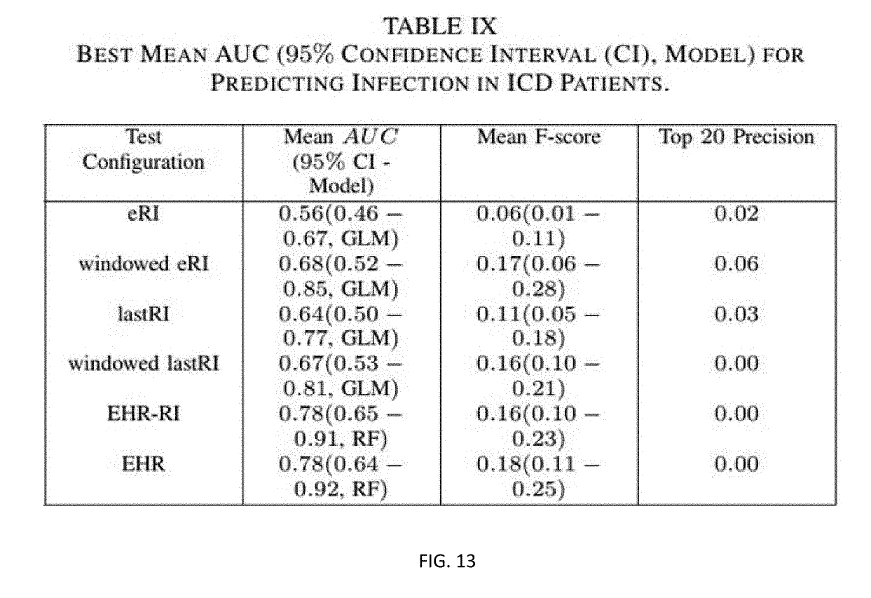

Results for the models developed for infection are presented in Table VII for CABG patients (see FIG. 11), Table VIII for PCI patients (see FIG. 12), and Table IX for ICD patients (see FIG. 13), respectively.

1) CABG Patients:

Models on CABG patients, in Table VII (see FIG. 11), using the windowed information of the Rothman Index did not provide the higher AUC, which was achieved by eRI at 0.67. Windowed eRI had the same AUC, however, provided a tighter confidence interval as well as provided a higher F-score at 0.41. The additional EHR data did not provide any improved AUC or F-score, and had a reduced top 20 precision of 0.00 down from 0.12. For the model with the highest F-score, the EHR model, the top features are listed here:
Fold 1: Prior History: Congestive Heart Failure—Unspecified, Present On Admission: Respiratory Failure, Present on Admission: Sepsis, Admit Source: Self Referral, Lab: INR
Fold 2: Prior History: Congestive Heart Failure—Unspecified, Lab: Anion Gap, Med: Solvents, Present On Admission: Respiratory Failure, Med: Heparin
Fold 3: Prior History: Unspecified Glaucoma, Primary Diagnosis: Unspecified Septicemia, Present On Admission: Respiratory Failure, Med: Sodium-Saline Preparations, Lab: Partial Thromboplastin Time is High Panic
Fold 4: Prior History: Congestive Heart Failure—Unspecified, Present On Admission: Respiratory Failure, Lab: PH UA is Abnormal, Lab RDW, Lab: Amorphous is Abnormal
Fold 5: Prior History: Congestive Heart Failure—Unspecified, Med: Sodium-Saline Preparations, Present On Admission: Respiratory Failure, Admit Source: Self-Referral, Present on Admission: Severe Sepsis

2) PCI Patients:

Models on PCI patients, presented in Table VIII (see FIG. 12), were able to better predict infection than in CABG patients or ICD patients. Similarly to CABG patients, using the earliest Rothman Index provided a higher AUC (0.72). In all cases, the use of EHR data provided significantly higher measurements from both the single model for PCI patients (0.67) and any of the Rothman Index test cases, with an AUC of 0.81 for EHR-RI and 0.83 for EHR, as well as an F-score of 0.12 and 0.14 respectively. The top 20 precision measurements were higher for PCI patients as well, as a measure of identifying high risk patients. For the model with the highest F-score, the EHR model, the top features are listed here:
Fold 1: Admission: Age, Med:Adrenergic Vasopressor Agents, Lab: Enterovirus by RT-PCR Stool is Abnormal, Lab: POC Activated Clotting Time is Abnormal, Med: Antihypertensives
Fold 2: Admission: Age, Lab: Albumin (EP) Urine Random is Abnormal, Med: Antivirals, Lab: Activated Protein C Resistance is Abnormal, Lab; Cortisol Plasma is Abnormal
Fold 3: Admission: Age, Lab: Fibrinogen Level, Lab: Vitamin D 25 Hydroxy is Abnormal, Lab: HCV Quantitative Log is Abnormal, Prior Coverage is Other
Fold 4: Admission: Age, Prior History: Acute Respiratory Failure, Lab: POC Appearance UA is Abnormal, Lab: Fluid Culture, Lab: POC Leukocytes UA is Abnormal
Fold 5: Admission: Age, Lab: Antibody Identification is Abnormal, Lab: Protein Creatinine Ratio Urine Random is Abnormal, Lab: Cocaine Screen Urine, Med: Folic Acid

3) ICD Patients: ICD patient infection predictions, presented in Table IX (see FIG. 13), were improved over the single model AUC of 0.67 from Table III (see FIG. 7). The Rothman Index models performed better than the single model case, as well, with the windowed eRI and windowed lastRI achieving AUC's of 0.68 and 0.67, respectively. Windowed eRI had the highest F-score of 0.17. The EHR-RI and EHR models performed the best, with the RF models achieving an AUC of 0.78 and 0.79, respectively and F-scores of 0.16 and 0.18, respectively. No model had top 20 precision. For the model with the highest F-score, the EHR model, the top features are listed here:
Fold 1: Primary Diagnosis: Combined Systolic and Diastolic Heart Failure—Acute on Chronic, Lab: Absolute Lymphocyte Count, Lab: Glucose Meter, Med: Sodium-Saline Preparations, Lab: International Normalization Ratio (POC)
Fold 2: Primary Diagnosis: Combined Systolic and Diastolic Heart Failure—Acute on Chronic, Lab: Bilirubin Total, Lab: Absolute Lymphocyte Count, Admit Source: Self Referral, Lab: Glucose Meter
Fold 3: Primary Diagnosis: Systolic Heart Failure—Acute on Chronic, Admit Source: Self Referral, Primary Diagnosis: Combined Systolic and Diastolic Heart Failure—Acute on Chronic, Med: Sodium-Saline Preparations, Lab: ECQ QT Interval
Fold 4: Primary Diagnosis: Systolic Heart Failure—Acute on Chronic, Admit Source: Self Referral, Primary Diagnosis: Combined Systolic and Diastolic Heart Failure—Acute on Chronic, Admit Source: Physician or Clinic Referral, Med: Sodium-Saline Preparations
Fold 5: Primary Diagnosis: Systolic Heart Failure—Acute on Chronic, Primary Diagnosis: Combined Systolic and Diastolic Heart Failure—Acute on Chronic, Lab: International Normalization Ratio POC, Admit Source: Self Referral, Admit Source: Physician or Clinic Referral

E. Calibration and Personalized Risk:

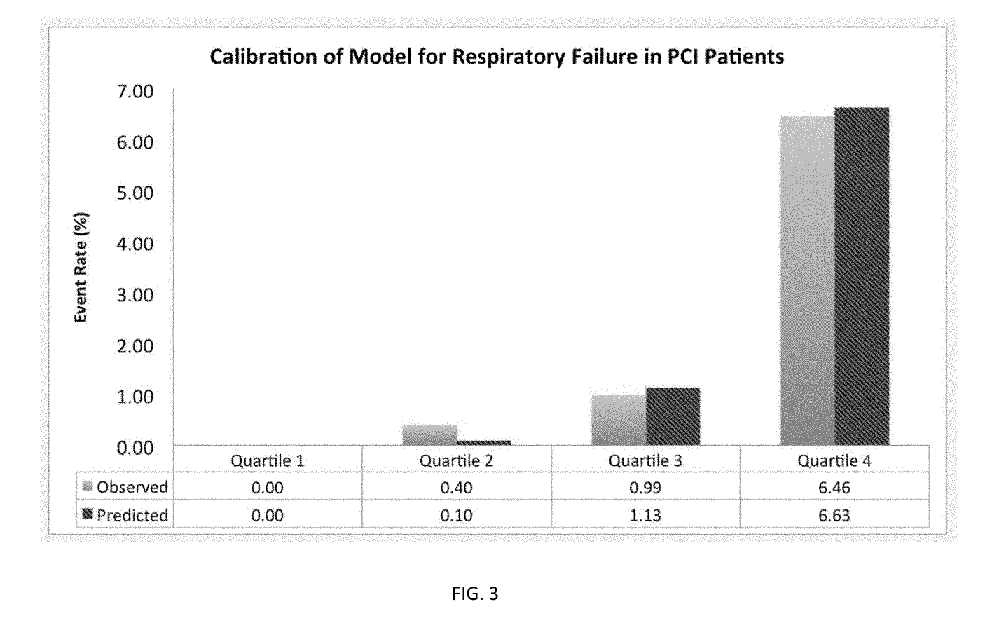

Understanding the factors behind the risk and outcome predicted is equally important to an accurate model. Thus, the system provided model calibration plots to better interpret patient risk. One such plot, for the model generating respiratory failure risk for PCI patients, is shown in FIG. 3. The calibration plot was created by sorting the probabilities generated by the model for the outcome into quartiles, then comparing the observed rate of respiratory failure to the mean risk for all predictions in each quartile. As shown in FIG. 3, quartile 1 has no observed respiratory failure predictions, thus, the high F-score of 0.25 and AUC of 0.81, despite the 0.00 Top 20 precision measure. This indicated that, while the model was able to generate a high risk group (quartile 4), the stratification within that group had room for improvement. Such calibration plots allow clinicians to better interpret the accuracy measurements generated by the models to understand underlying risk.

Further, along with the generated model accuracy, predictions, and calibration plots, the important features that generate the risk for a given patient were important in determining a cause and potential intervention. While each method provided a global list of important features, how each feature contributes to an individual's total risk score should be understood. Thus, the system generates an identification of which risk quartile the patient lies within, as well as the personalized response to the GLM model, as detailed in Section I-F. As an illustrative example, the GLM for the PCI respiratory failure, which achieved a mean AUC of 0.76 used the following features:
Lab 1—Blood Urea Nitrogen is High −β=0.0910
Lab 2—Anion Gap is High −β=0.1124
Med 1—Anti-Hyperlipidemic—HMG COA Reductase Inhibitors Given −β=0.0142
Primary Diagnosis—Coronary atherosclerosis of native coronary artery −β=0.2751

Consider the following two patient \vec{feat} vectors. The patient risk for patient X₁ was 0.61 while the patient risk for patient X₂ was 0.62. Both patients did, indeed, have respiratory failure, as correctly indicated by the model. However for X₁, \vec{feat(x₃)}= [symbol] 0.273, 0.337, −0.014, 0 [symbol] while for X₂, \vec{feat(x₂)}= [symbol] 0.273, 0.337, −0.028, 0 [symbol] . This specific level of information illustrated the top contributors to the patient's specific risk score were, which could be extremely important in cases where the models might select hundreds of variables. In this particular case, the second patient had had more medication than the first, slightly increasing the predicted risk.

Section III. Discussion

A. Single Model Results:

The results showed an interesting distribution of strengths and areas of necessary improvement. Having all patients together confounded the results, achieving low AUCs despite the methods employed and high top 20 precision. The added data did not appear to help for most patients. Thus, such settings were only ideal for identifying those at highest risk. Table III (see FIG. 7) shows that evaluating each group individually lead to a better understanding of strengths and weaknesses. In particular, PCI and ICD patients improved over the all patients model, while CABG patients were reduced. In some instances, those individual CABG patients can be better predicted by the all patients model, but it is likely that they were similarly missed there. Thus, separating models into individual ones for each patient group achieved greater success, enabling more specific results in future interventions. The system used the best available model knowing the particular patient.

B. Cohort-Specific Features and Results:

For the respiratory failure and infection models, significant improvement was seen in the PCI patients and ICD patients. These models saw significant improvement by separating out the patient cohorts as well as incorporating the spectrum of EHR data selected. In these cases, the Rothman Index tests, with fewer variables, were well modeled by GLM, while RF and XGB provided the higher accuracy when the significantly wider array of variables was provided. In many cases, the EHR-RI and EHR models performed similarly. The Rothman Index provided some added value, but in all cases, the extension of the datasets to the EHR data provided the largest basis for improvement. As more features were added to the models, and the complexity increased, the non-linear, non-parametric methods were better suited to finding higher-dimensional patterns for prediction. This became quite apparent when looking at the top features selected for each model in each fold. The GLM models, best in CABG patients, selected mostly binary variables. In contrast, the RF and XGB models often chose continuous variables, and a spread of medication information, laboratory results, as well as prior history and patient presentation information. The reference value flags were often selected as well, which aligns thinking with clinical interpretability. Of note was that the top selected features for XGB were a majority of numeric laboratory results, rather than the flag values of the labs selected by RF and GLM. Further, the present on admission flags along with laboratory values for these tree-based methods may have allowed for the removal of a number of false positives, thus improving AUC and F-score (improved recall) but not top 20 precision.

The numeric results for AUC, F-score and top 20 also aligned with calibration results. In particular, the improved AUC values indicated a better opportunity for the models to discriminate patients. With the low AUCs in CABG, all following results were similarly low, because an effective threshold delineating adverse outcomes and healthy outcomes was not clear. The lower F-scores, with the improved AUCs, were a function of the event rate. The low score indicated that the recall (sensitivity) was high but the precision was low. So while the threshold for determining clearly healthy outcomes was well-established, the mix of true positive predictions and false positive predictions is still an area for further investigation. This was also demonstrated in the top 20 precision and the calibration results. The right-skewed calibration results indicated that the adverse outcomes were mostly in the highest quartile of risk. However, with the low top 20 precision, these patients were not the highest risk. An expansion of the binary outcomes to multiple classes, with tiered understandings of the postoperative period, might be necessary to understand these false positive patients and why they are predicted differently than the large number of correctly identified true negative patients. This may also be because of other events that are not currently recorded or considered adverse outcomes in this study.

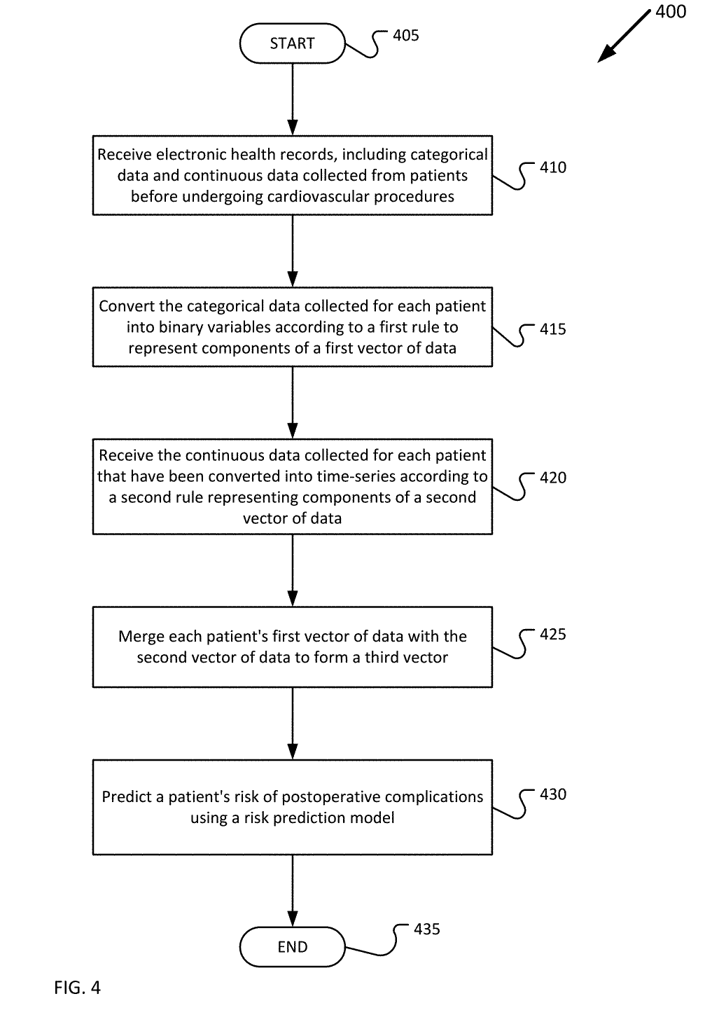

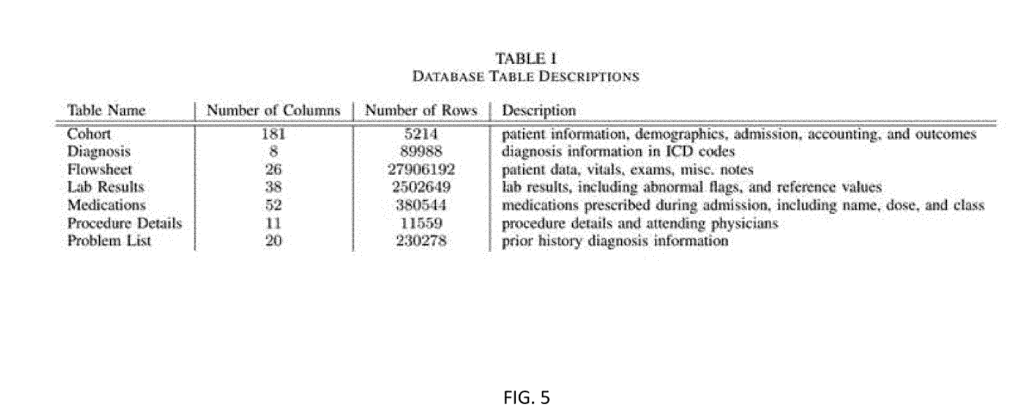

FIG. 4 shows an example process 400 for identifying patients undergoing cardiovascular procedures at risk for postoperative complications. The process 400 starts (405) and receives (410) electronic health records (ERH) stored e.g., in an ERH database (a representation of which is provided in FIG. 1). The ERH include categorical data and continuous data collected from patients before they undergo cardiovascular procedures. The process 400 converts (415) the categorical data collected for each patient into binary variables according to a first rule.

An example rule for converting data related to medication or drugs, which are prescribed to a patient, into binary variables can include removing dosage information from the data. This is done because many of the drug dosages are standard, e.g., 325 mg of aspirin. This conversion step is beneficial for machine learning techniques because data broken down by medication dosage and by delivery type tend to be sparse. Sparse data is a common problem in machine learning, which alters the performance of machine learning algorithms and their ability to calculate accurate predictions. Data is considered sparse when certain expected values in a dataset are missing, which is a common phenomenon in general large scaled data analysis

With the dosages information removed from the data relating to the prescribed drugs, the number of drugs that a patient has at different doses is then added up resulting in an integer variable. Additionally or separately, the drug-related data can also be combined by medical class that is either defined by clinician or by pharmaceutical class. Again adding up these binary variables results in an integer variable for how many of these types of drugs the patient has been prescribed.

An example rule for converting data related to labs into binary variables includes using data from the last lab drawn. For example, the lab name drawn can be converted into a “yes” or “no”. If the lab is drawn, then the value of the lab is recorded. If the lab value is missing then a “−1” or some other marker is recorded. When the lab is drawn, the lab flag is recorded as either “normal” or “abnormal”. When the lab is not drawn, then the lab flag is recorded as “not drawn”. The foregoing binary variables can be combined to form a vector representing the lab-related data for particular patient.

Returning the process 400, the binary variables represent components of a first vector of data have a first vector length. The resulting vectors each have the same vector length regardless of how much data was collected from the patient. For example, Patient 1 is only in the hospital for three hours before his operation and one vital reading was taken. In contrast, Patient 2 is in the hospital for 48 hours before her operation and has her blood pressure taken every four hours for a total of twelve vital readings. The data related to the vitals from these two patients are different, viz, Patient 1 has one vital reading while with Patient 2 has twelve vital readings.

To be useful in predicting a patient's risk of a postoperative complication, each patient's blood pressure, for example, is described by transforming all the individual systolic blood pressure (sbp) readings to: mean sbp, standard deviation of sbp, min of sbp, max of sbp, and number of sbp readings. In this way, data relating to Patient 1's blood pressure and Patient 2's blood pressure are converting into vectors each having the same vector length, viz., five variables long, despite the differences in the number of vitals reading that are actually taken.

The process 400 receives (420) the continuous data collected for each patient that has been converted into time-series according to a second rule. The second rule is different than the first rule. In another example, the process 400 converts (not shown) the continuous data collected for each patient into the time-series according to the second rule.

The blood pressure example provided above is an example of a rule for converting continuous data collected for each patient into time-series. The time-series represent components of a second vector of data have a second vector length. The second vector length of each patient's second vector of data is the same. In the blood pressure example above, the vectors representing the blood pressure data for Patient 1 and Patient 2 are both five variables long despite the difference in the number of vitals taken from the patients. Some examples of continuous data, such as age, are not in a time series.

The process 400 then merges (425) each patient's first vector of data with the second vector of data to form a third vector of data. The third vector of data has a third vector length. The third vector length of each patient's third vector of data is the same. While the described above as processing categorical data and continuous data, the process 400 can also handle other types of data. For example, the process 400 can be provided with data relating to a digital image. The process 400 converts the data into a single row vector of pixels by appending each horizontal row if pixels to each other. In a convenient example, features of interest are extracted from the digital image and then the result is converted into a vector. This pre-processing is advantageous because it can reduce the amount of data to be processed into a vector, and thus decrease the amount of computing power needed and/or decrease the amount of computing time needed.

The process 400 predicts (430) a patient's risk of postoperative complications using a risk prediction model and the process 400 ends (4350.

A convenient example of the invention includes model generation process (not shown) for generating the risk prediction model using preoperative categorical and continuous data collected from prior (earlier) patients who underwent the same procedure as the present (current) patient. The process model generation process includes converting each prior patient's continuous data into training binary variables according to the first rule (used in the prediction process 400 above). The training binary variables represent components of a fourth vector of data having a fourth vector length. The fourth vector length associated with each prior patient and the first vector length associated with the present patient are the same.

The model generation process further includes receiving each prior patient's continuous data converted into training time-series according to the second rule (used in the prediction process 400 above). The training time-series represent components of a fifth vector of data having a fifth vector length. The fifth vector length associated with each prior patient and the second vector length associated with the present patient are the same. The model generation further includes merging each prior patient's fourth vector of data with the fifth vector of data to form a sixth vector of data having a sixth vector length. The sixth vector length associated with each prior patient and the third vector length associated with the present patient are the same.

The model generation process further includes generating a training dataset based on the sixth vector of data of each prior patient and applying a machine learning technique to the training dataset to generate the risk prediction model. The machine learning technique that is applied can be generalized linear model, random forest machine learning or gradient descent boosting.

The process 400 generates (430) a training dataset based on the third vector of data of each patient. The process 400 applies (435) a machine learning technique to the training dataset to generate a risk prediction model. The process 400 predicts a patient's risk of postoperative complications using the risk prediction model. The foregoing process 400 can be coded as instructions that are stored in a non-transitory computer readable medium and the instructions can be executed by a processor.

The above-described systems and methods can be implemented in digital electronic circuitry, in computer hardware, firmware, and/or software. The implementation can be as a computer program product. The implementation can, for example, be in a machine-readable storage device, for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by and an apparatus can be implemented as special purpose logic circuitry. The circuitry can, for example, be a FPGA (field programmable gate array) and/or an ASIC (application-specific integrated circuit). Subroutines and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can include, can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device. The display device can, for example, be a cathode ray tube (CRT) and/or a liquid crystal display (LCD) monitor. The interaction with a user can, for example, be a display of information to the user and a keyboard and a pointing device (e.g., a mouse or a trackball) by which the user can provide input to the computer (e.g., interact with a user interface element). Other kinds of devices can be used to provide for interaction with a user. Other devices can, for example, be feedback provided to the user in any form of sensory feedback (e.g., visual feedback, auditory feedback, or tactile feedback). Input from the user can, for example, be received in any form, including acoustic, speech, and/or tactile input.

The above described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, bluetooth, code-division multiple access (CDMA) network, time division multiple access (TDMA) network, global system for mobile communications (GSM) network), and/or other circuit-based networks.

The transmitting device can include, for example, a computer, a computer with a browser device, a telephone, an IP phone, a mobile device (e.g., cellular phone, personal digital assistant (PDA) device, laptop computer, electronic mail device), and/or other communication devices. The browser device includes, for example, a computer (e.g., desktop computer, laptop computer) with a world wide web browser (e.g., Microsoft® Internet Explorer® available from Microsoft Corporation, Mozilla® Firefox available from Mozilla Corporation). The mobile computing device includes, for example, a Blackberry®.

Comprise, include, and/or plural forms of each are open ended and include the listed parts and can include additional parts that are not listed. And/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

### REFERENCES

[1] K. Rahimi, D. Bennett, N. Conrad, T. M. Williams, J. Basu, J. Dwight, M. Woodward, A. Patel, J. McMurray, and S. MacMahon, “Risk prediction in patients with heart failure: a systematic review and analysis,” JACC: Heart Failure, vol. 2, no. 5, pp. 440-446, 2014.
[2] J. S. Ross, G. K. Mulvey, B. Stauffer, V. Patlolla, S. M. Bernheim, P. S. Keenan, and H. M. Krumholz, “Statistical models and patient predictors of readmission for heart failure: a systematic review,” Archives of internal medicine, vol. 168, no. 13, pp. 1371-1386, 2008.
[3] B. B. Dean, J. Lam, J. L. Natoli, Q. Butler, D. Aguilar, and R. J. Nordyke, “Review: Use of electronic medical records for health out-comes research a literature review,” Medical Care Research and Review, vol. 66, no. 6, pp. 611-638, 2009.
[4] S. Saria, A. K. Rajani, J. Gould, D. Koller, and A. A. Penn, “Integration of early physiological responses predicts later illness severity in preterm infants,” Science translational medicine, vol. 2, no. 48, pp. 48ra65-48ra65, 2010.
[5] J. P. Fischer, A. M. Wes, J. D. Wink, J. A. Nelson, B. M. Braslow, and S. J. Kovach, “Analysis of risk factors, morbidity, and cost associated with respiratory complications following abdominal wall reconstruction,” Plastic and reconstructive surgery, vol. 133, no. 1, pp. 147-156, 2014.
[6] G. J. Murphy, B. C. Reeves, C. A. Rogers, S. I. Rizvi, L. Culliford, and G. D. Angelini, “Increased mortality, postoperative morbidity, and cost after red blood cell transfusion in patients having cardiac surgery,” Circulation, vol. 116, no. 22, pp. 2544-2552, 2007.
[7] R. Amarasingham, B. J. Moore, Y. P. Tabak, M. H. Drazner, C. A. Clark, S. Zhang, W. G. Reed, T. S. Swanson, Y. Ma, and E. A. Halm, “An automated model to identify heart failure patients at risk for 30-day readmission or death using electronic medical record data,” Medical care, vol. 48, no. 11, pp. 981-988, 2010.
[8] K. E. Henry, D. N. Hager, P. J. Pronovost, and S. Saria, “A targeted real-time early warning score (trewscore) for septic shock,” Science Translational Medicine, vol. 7, no. 299, pp. 299ra122-299ra122, 2015.
[9] J. A. Rubano, J. A. Vosswinkel, J. E. McCormack, E. C. Huang, M. J. Shapiro, and R. S. Jawa, “Unplanned intensive care unit admission following trauma,” Journal of Critical Care, vol. 33, pp. 174-179, 2016.
[10] M. Bayati, M. Braverman, M. Gillam, K. M. Mack, G. Ruiz, M. S. Smith, and E. Horvitz, “Data-driven decisions for reducing readmissions for heart failure: General methodology and case study,” PloS one, vol. 9, no. 10, p. e109264, 2014.
[11] A. Visser, B. Geboers, D. J. Gouma, J. C. Goslings, and D. T. Ubbink, Predictors of surgical complications: A systematic review,” Surgery, vol. 158, no. 1, pp. 58-65, 2015.
[12] M. Bayati, S. Bhaskar, and A. Montanari, “A low-cost method for multiple disease prediction,” in AMIA Annual Symposium Proceedings, vol. 2015. American Medical Informatics Association, 2015, p. 329
[13] “Strata partners with yale new haven health system to reduce cost by improving quality,” http://www.stratadecision.com/our-company/newsroom/press-releases/2015/04/10/strata-partners-with-yale-new-haven-health-system-to-reduce-cost\\-by-improving-quality, accessed: 2016 May 2023.
[14] R. Palmerola, C. Hartman, N. Theckumparampil, A. Mukkamala, J. Fishbein, M. Schwartz, and L. R. Kavoussi, “Surgical complications and their repercussions,” Journal of Endourology, vol. 30, no. 51, pp. S-2, 2016.
[15] A. Güldner, P. M. Spieth, and M. G. de Abreu, “Non-ventilatory approaches to prevent postoperative pulmonary complications,” Best Practice & Research Clinical Anaesthesiology, vol. 29, no. 3, pp. 397-410, 2015.
[16] G. Ottino, R. De Paulis, S. Pansini, G. Rocca, M. V. Tallone, C. Comoglio, P. Costa, F. Orzan, and M. Morea, “Major sternal wound infection after open-heart surgery: a multivariate analysis of risk factors in 2,579 consecutive operative procedures,” The Annals of thoracic surgery, vol. 44, no. 2, pp. 173-179, 1987.
[17] L. Gallart and J. Canet, “Post-operative pulmonary complications: Understanding definitions and risk assessment,” Best Practice & Research Clinical Anaesthesiology, vol. 29, no. 3, pp. 315-330, 2015.
[18] G. Luc, M. Durand, L. Chiche, and D. Collet, “Major post-operative complications predict long-term survival after esophagectomy in patients with adenocarcinoma of the esophagus,” World journal of surgery, vol. 39, no. 1, pp. 216-222, 2015.
[19] S. N. Hemmes, A. S. Neto, and M. J. Schultz, “Intraoperative ventilatory strategies to prevent postoperative pulmonary complications: a meta-analysis,” Current Opinion in Anesthesiology, vol. 26, no. 2, pp. 126-133, 2013.
[20] R. G. Johnson, A. M. Arozullah, L. Neumayer, W. G. Henderson, P. Hosokawa, and S. F. Khuri, “Multivariable predictors of postoperative respiratory failure after general and vascular surgery: results from the patient safety in surgery study,” Journal of the American College of Surgeons, vol. 204, no. 6, pp. 1188-1198, 2007.
[21] R. H. Mehta, J. D. Grab, S. M. OBrien, C. R. Bridges, J. S. Gammie, C. K. Haan, T. B. Ferguson, E. D. Peterson, S. of Thoracic Surgeons National Cardiac Surgery Database Investigators et al., “Bedside tool for predicting the risk of postoperative dialysis in patients undergoing cardiac surgery,” Circulation, vol. 114, no. 21, pp. 2208-2216, 2006.
[22] I. K. Toumpoulis, C. E. Anagnostopoulos, D. G. Swistel, and J. J. DeRose, “Does euroscore predict length of stay and specific postoperative complications after cardiac surgery?” European journal of cardio-thoracic surgery, vol. 27, no. 1, pp. 128-133, 2005.
[23] H. F. Elkhenini, K. J. Davis, N. D. Stein, J. P. New, M. R. Delderfield, M. Gibson, J. Vestbo, A. Woodcock, and N. D. Bakerly, “Using an electronic medical record (emr) to conduct clinical trials: Salford lung study feasibility,” BMC medical informatics and decision making, vol. 15, no. 1, p. 1, 2015.
[24] R. Amarasingham, A.-M. J. Audet, D. W. Bates, I. G. Cohen, M. Entwistle, G. Escobar, V. Liu, L. Etheredge, B. Lo, L. Ohno-Machado et al., “Consensus statement on electronic health predictive analytics: A guiding framework to address challenges,” eGEMs, vol. 4, no. 1, 2016.
[25] R. Miotto, L. Li, B. A. Kidd, and J. T. Dudley, “Deep patient: An unsupervised representation to predict the future of patients from the electronic health records,” Scientific Reports, vol. 6, p. 26094, 2016.
[26] P. Schulam, F. Wigley, and S. Saria, “Clustering longitudinal clinical marker trajectories from electronic health data: Applications to phenotyping and endotype discovery.” in AAAI, 2015, pp. 2956-2964.
[27] S. Saria and A. Goldenberg, “Subtyping: What it is and its role in precision medicine,” Intelligent Systems, IEEE, vol. 30, no. 4, pp. 70-75, 2015.
[28] J. Wiens, W. N. Campbell, E. S. Franklin, J. V. Guttag, and E. Horvitz, “Learning data-driven patient risk stratification models for clostridium difficile,” in Open forum infectious diseases, vol. 1, no. 2. Oxford University Press, 2014, p. ofu045.
[29] M. J. Rothman, S. I. Rothman, and J. Beals, “Development and validation of a continuous measure of patient condition using the electronic medical record,” Journal of biomedical informatics, vol. 46, no. 5, pp. 837-848, 2013.
[30] G. D. Finlay, M. J. Rothman, and R. A. Smith, “Measuring the modified early warning score and the rothman index: advantages of utilizing the electronic medical record in an early warning system,” Journal of hospital medicine, vol. 9, no. 2, pp. 116-119, 2014.
[31] E. Bradley, O. Yakusheva, L. I. Horwitz, H. Sipsma, and J. Fletcher, “Identifying patients at increased risk for unplanned readmission,” Medical care, vol. 51, no. 9, p. 761, 2013.
[32] G. L. Piper, L. J. Kaplan, A. A. Maung, F. Y. Lui, K. Barre, and K. A. Davis, “Using the rothman index to predict early unplanned surgical intensive care unit readmissions,” Journal of Trauma and Acute Care Surgery, vol. 77, no. 1, pp. 78-82, 2014.
[33] “Epic electronic medical record,” http://www.epic.com/.
[34] “Strata qvi,” http://www.stratadecision.com/qvi.
[35] R. G. Brindis, S. Fitzgerald, H. V. Anderson, R. E. Shaw, W. S. Weintraub, and J. F. Williams, “The american college of cardiology-national cardiovascular data registry(acc-ncdr): building a national clinical data repository,” Journal of the American College of Cardiology, vol. 37, no. 8, pp. 2240-2245, 2001.
[36] J. Friedman, T. Hastie, and R. Tibshirani, “Regularization paths for generalized linear models via coordinate descent,” Journal of Statistical Software, vol. 33, no. 1, pp. 1-22, 2010. [Online]. Available: http://www.jstatsoft.org/v33/i01/[37]
[37] A. Liaw and M. Wiener, “Classification and regression by randomforest,” R News, vol. 2, no. 3, pp. 18-22, 2002. [Online]. Available: http://CRAN.R-proj ect.org/doc/Rnews/[38]
[38] T. Chen and C. Guestrin, “Xgboost: A scalable tree boosting system,” arXiv preprint arXiv: 1603.02754, 2016. T. Fawcett, “An introduction to roc analysis,” Pattern recognition letters, vol. 27, no. 8, pp. 861-874, 2006.

What is claimed is:

1. A method for predicting a patient's risk of a postoperative complication from a procedure, the method comprising:
receiving, by a system comprising a processor, electronic health records stored in memory, the electronic health records include preoperative categorical and continuous data collected from a present patient before undergoing a procedure;
converting, by the system, the preoperative categorical data into binary variables according to a first rule, wherein the binary variables represent components of a first vector of data having a first vector length;
receiving, by the system, the preoperative continuous data converted into a time-series according to a second rule different than the first rule, wherein the time-series represent components of a second vector of data having a second vector length;
merging, by the system, the present patient's first and second vectors of data to form a third vector of data having a third vector length; and
predicting, by the system, the present patient's risk of a postoperative complication from the procedure based on the third vector using a risk prediction model.

2. The method of claim 1, wherein receiving the electronic health records includes receiving the preoperative categorical and continuous data that has been collected from the present patient over a 24 hour period starting from when the present patient is admitted.

3. The method of claim 1, wherein the present patient's preoperative categorical data include any one of age, gender, insurance, admission information, patient problem list, admission diagnosis codes, primary principal procedure information, admission time, attending staff information, medications prescribed, medical images or combinations thereof.

4. The method of claim 1, wherein the present patient's preoperative categorical data includes medication prescribed to the patient before undergoing the procedure and dosage; and wherein converting the preoperative categorical data into binary variables according to the first rule includes when the prescribed medication is two or more dosages of the same medication, then combining binary variables describing each of the dosages into an integer variable having a value equal to the number of dosages.

5. The method of claim 1, wherein the present patient's preoperative categorical data includes medication prescribed to the patient before undergoing the procedure and dosage; and wherein converting the preoperative categorical data into binary variables according to the first rule includes when the prescribed medication is two or more different medications belonging to the same class of medications, then combining binary variables describing each of the different medications into an integer variable having a value equal to the number of the different medications.

6. The method of claim 1, wherein the present patient's preoperative categorical data includes medication prescribed to the patient before undergoing the procedure and dosage; and wherein converting the categorical data into binary variables according to the first rule includes when the prescribed medication is two or more different medications belonging to different classes of medications, then describing each of the different medications as a binary variable having a value of 1.

7. The method of claim 1, wherein the present patient's preoperative continuous data include any one of laboratory results, vital readings, temperature, pulse oxygenation, systolic blood pressure, diastolic blood pressure, respiratory rate, heart rate, Rothman index scores or combinations thereof.

8. The method of claim 1, wherein the preoperative continuous data includes the present patient's vital readings taken before undergoing the procedure, and wherein converting the preoperative continuous data into the time-series according to the second rule includes setting a first variable of the time-series to the mean of the vital readings and setting a second variable of the time-series to the standard deviation of the vital readings.

9. The method of claim 1, wherein the preoperative continuous data includes the present patient's vital readings taken before undergoing the procedure, and wherein converting the preoperative continuous data into the time-series according to the second rule includes setting a first variable of the time-series to the first vital reading taken and setting a second variable of the time-series to the last vital reading taken.

10. The method of claim 1, wherein the preoperative continuous data includes the present patient's laboratory results from tests performed before undergoing the procedure, and wherein converting the preoperative continuous data into the time-series according to the second rule includes setting a first variable of the time-series to the mean of the laboratory results and setting a second variable of the time-series to the standard deviation of the laboratory results.

11. The method of claim 1, wherein the risk prediction model includes a threshold determined from a receiver operating characteristic (ROC) analysis of the risk prediction model; and wherein predicting the present patient's risk of a postoperative complication includes generating a risk prediction by running the risk prediction model on the present patient's third vector of data, the components of which represent preoperative categorical and continuous data collected for the patient before undergoing the procedure; comparing the risk prediction to the threshold; and determining whether the present patient is at risk of postoperative complications based on the comparison.

12. The method of claim 1, further comprising normalizing the binary variables and the time-series.

13. The method of claim 1, wherein a time-series is missing a value, the method further comprising replacing the missing value with any one a mean value and a median value.

14. The method of claim 1, further comprising generating the risk prediction model by the system, the model generation comprises: receiving electronic health records including preoperative categorical and continuous data collected from prior patients who underwent the same procedure as the present patient;
converting each prior patient's continuous data into training binary variables according to the first rule, wherein the training binary variables represent components of a fourth vector of data having a fourth vector length, and wherein the fourth vector length associated with each prior patient and the first vector length associated with the present patient are the same;
receiving each prior patient's continuous data converted into training time-series according to the second rule, wherein the training time-series represent components of a fifth vector of data having a fifth vector length, and wherein the fifth vector length associated with each prior patient and the second vector length associated with the present patient are the same;
merging each prior patient's fourth vector of data with the fifth vector of data to form a sixth vector of data having a sixth vector length, wherein the sixth vector length associated with each prior patient and the third vector length associated with the present patient are the same;
generating a training dataset based on the sixth vector of data of each prior patient; and
applying a machine learning technique to the training dataset to generate the risk prediction model.

15. The method of claim 14, wherein applying the machine learning technique includes applying any one of generalized linear model and random forest machine learning techniques.

16. The method of claim 14, further comprising validating the risk prediction model with a five-fold validation.

17. A non-transitory computer readable medium storing instructions which, when executed by a system comprising a processor, cause the processor to perform operations comprising:
receiving electronic health records stored in memory, the electronic health records include preoperative categorical and continuous data collected from a present patient before undergoing a procedure;
converting the preoperative categorical data into binary variables according to a first rule, wherein the binary variables represent components of a first vector of data having a first vector length;
receiving the preoperative continuous data converted into a time-series according to a second rule different than the first rule, wherein the time-series represent components of a second vector of data having a second vector length;
merging the present patient's first and second vectors of data to form a third vector of data having a third vector length; and
predicting the present patient's risk of a postoperative complication from the procedure based on the third vector using a risk prediction model.

18. A system comprising: a processor; and a memory that stores instructions that, when executed by the processor, cause the processor to perform operations comprising:
receiving electronic health records stored in memory, the electronic health records include preoperative categorical and continuous data collected from a present patient before undergoing a procedure;
converting the preoperative categorical data into binary variables according to a first rule, wherein the binary variables represent components of a first vector of data having a first vector length;
receiving the preoperative continuous data converted into a time-series according to a second rule different than the first rule, wherein the time-series represent components of a second vector of data having a second vector length;
merging the present patient's first and second vectors of data to form a third vector of data having a third vector length; and
predicting the present patient's risk of a postoperative complication from the procedure based on the third vector using a risk prediction model.
